(19) United States
(12) Patent Application Publication
(10) Pub. No.: US 2026/0220440 A1
(43) Pub. Date: Jul. 30, 2026

(54) METHOD AND DEVICE FOR PREDICTING YIELD

(71) Applicants: SAMSUNG ELETRONICS CO., LTD., Suwon-si (KR); RESEARCH & BUSINESS FOUNDATION SUNGKYUNKWAN UNIVERSITY, Suwon-si (KR)

(72) Inventors: Youngchun KWON, Suwon-si (KR); Seokho Kang, Gwacheon-si (KR); Yongsik Jung, Suwon-si (KR)

(73) Assignees: SAMSUNG ELETRONICS CO., LTD., Suwon-si (KR); RESEARCH & BUSINESS FOUNDATION SUNGKYUNKWAN UNIVERSITY, Suwon-si (KR)

(21) Appl. No.: 19/439,043

(22) Filed: Jan. 2, 2026

(30) Foreign Application Priority Data
Jan. 24, 2025 (KR) 10-2025-0010804
Mar. 20, 2025 (KR) 10-2025-0035866

(51) Int. Cl. G06N 3/0475; G06N 3/045

(52) U.S. Cl. CPC G06N 3/0475; G06N 3/045

(57) ABSTRACT

A method of predicting yield may include receiving input data including a product and reaction factors, obtaining reaction factor embedding vectors via an encoder based on the reactor factors, obtaining a product embedding vector via the encoder based on the product, obtaining a reaction embedding vector corresponding to an entire reaction via a projection layer based on the reaction factor embedding vectors and the product embedding vector, predicting a contribution of each of the reaction factors based on the reaction embedding vector and the reaction factor embedding vectors, and predicting yield of the entire reaction based on the contribution of each of the reaction factors.

### CROSS-REFERENCE TO RELATED APPLICATION

This application claims priority from Korean Patent Application No. 10-2025-0010804, filed on Jan. 24, 2025, and Korean Patent Application No. 10-2025-0035866, filed on Mar. 20, 2025, in the Korean Intellectual Property Office, the entire disclosure of which are incorporated herein by reference for all purposes.

### BACKGROUND

### 1. Field

Embodiments are related to a method and device for predicting yield, and more particularly, to a method of predicting interpretable yield.

### 2. Description of the Related Art

Research has been actively conducted across industries to optimize processes and improve productivity. Particularly, a method of adjusting a process condition to obtain a desired result has been addressed as an important issue in various fields, such as material synthesis, reaction processes, and manufacturing processes.

Typically, a reaction factor or a process variable may have a direct impact on the quality and yield of a final product. Accordingly, a method of optimizing a combination of reaction factors has been studied through experiments, and experimental approaches or simulation techniques are widely used.

Recently, research for learning and predicting a relation between a process variable and a result increases by using machine learning (ML) and data-driven optimization.

The above information may be presented as the related art to help with the understanding of the disclosure. No arguments or decisions are raised as to whether any of the above description is applicable as the prior art related to the present disclosure.

### SUMMARY

One or more embodiments may address at least the above problems and/or disadvantages and other disadvantages not described above. Also, the embodiments are not required to overcome the disadvantages described above, and an embodiment may not overcome any of the problems described above.

According to an aspect of an embodiment, a method of predicting yield may include: receiving input data including a product and reaction factors; obtaining reaction factor embedding vectors via an encoder based on the reactor factors; obtaining a product embedding vector via the encoder based on the product; obtaining a reaction embedding vector corresponding to an entire reaction via a projection layer based on the reaction factor embedding vectors and the product embedding vector; predicting a contribution of each of the reaction factors based on the reaction embedding vector and the reaction factor embedding vectors; and predicting yield of the entire reaction based on the contribution of each of the reaction factors.

The predicting of the contribution of each of the reaction factors may include predicting the contribution of each of the reaction factors by inputting the reaction embedding vector and the reaction factor embedding vectors to a prediction head.

The predicting of the yield of the entire reaction may include predicting the yield of the entire reaction by summing the contributions of the reaction factors.

The encoder may include a graph neural network (GNN) encoder, the reaction factor may include a precursor and a reaction condition, and the obtaining of the reaction factor embedding vectors may include converting the precursor into a precursor embedding vector by inputting the precursor to the GNN encoder in a graph form.

The encoder may include a fully connected neural network (FNN) encoder, the obtaining of the reaction factor embedding vectors may include converting the reaction condition into a reaction condition embedding vector by inputting the reaction condition to the FNN encoder, and the reaction condition embedding vector and the precursor embedding vector are set to a same size.

The obtaining of the product embedding vector may include converting the product into the product embedding vector by inputting the product to the GNN encoder.

The obtaining of the reaction embedding vector may include obtaining a concatenated reaction factor embedding vectors by concatenating the reaction factor embedding vectors with the product embedding vector and inputting the concatenated the reaction factor embedding vectors with the product embedding vector to the projection layer.

The entire reaction may include at least one of a chemical reaction, an electrochemical reaction, a biochemical reaction, a material conversion process, a physical processing process, a data conversion process, or a manufacturing process.

The method may include: predicting the contribution of each of the reaction factors and the yield with respect to a plurality of reaction factor combinations by differently setting the reaction factors; and determining a reaction factor according to a predefined criterion based on a predicted contribution and yield with respect to the plurality of reaction factor combinations.

According to an aspect of an embodiment, there is provided a method of training including receiving training data including a product and reaction factors, converting each of the reaction factors into a reaction factor embedding vector by inputting each of the reaction factors to an encoder of a model, converting the product into a product embedding vector by inputting the product to the encoder, generating a reaction embedding vector corresponding to an entire reaction by inputting the reaction factor embedding vector and the product embedding vector to a projection layer of the model, predicting a contribution of each of the reaction factors by inputting the reaction embedding vector and the reaction factor embedding vector to a prediction neural network of the model, predicting yield of the entire reaction based on the contribution of each of the reaction factors, and updating a weight of the model based on the predicted yield.

The updating of the weight of the model includes updating the weight of the model based on a first loss function that minimizes a difference between the predicted yield and ground truth yield, a second loss function that normalizes a sum of the contributions of the reaction factors to satisfy a predefined criterion, and a third objective function that corrects a contribution of a reaction factor that does not affect the yield.

According to an aspect of an embodiment, there is provided an electronic device including at least one processor including processing circuitry, and memory storing instructions, wherein the instructions, when executed individually or collectively by the at least one processor, cause the electronic device to receive input data including a product and reaction factors, obtain reaction factor embedding vectors via an encoder based on the reactor factors, obtain a product embedding vector via the encoder based on the product, obtain a reaction embedding vector corresponding to an entire reaction via a projection layer based on the reaction factor embedding vectors and the product embedding vector, predict a contribution of each of the reaction factors based on the reaction embedding vector and the reaction factor embedding vectors, and predict yield of the entire reaction based on the contribution of each of the reaction factors.

The instructions, when executed individually or collectively by the at least one processor, cause the electronic device to predict the contribution of each of the reaction factors by inputting the reaction embedding vector and the reaction factor embedding vectors to a prediction head.

Each sentence includes a plurality of feature vectors, and the instructions, when executed individually or collectively by the at least one processor, cause the electronic device to predict the yield of the entire reaction by summing the contributions of the reaction factors.

The encoder may include a fully connected neural network (FNN) encoder, and the reaction factor includes a precursor and a reaction condition, and the instructions, when executed individually or collectively by the at least one processor, cause the electronic device to convert the reaction condition into a reaction condition embedding vector by inputting the reaction condition to FNN encoder.

The instructions, when executed individually or collectively by the at least one processor, cause the electronic device to convert the reaction condition into the reaction condition embedding vector by inputting the reaction condition to the FNN encoder.

The instructions, when executed individually or collectively by the at least one processor, cause the electronic device to convert the product into the product embedding vector by inputting the product to a graph neural network (GNN) encoder in a graph form.

The instructions, when executed individually or collectively by the at least one processor, cause the electronic device to generate the reaction embedding vector by concatenating the reaction factor embedding vector with the product embedding vector and inputting the concatenated the reaction factor embedding vector with the product embedding vector to the projection layer.

The entire reaction includes at least one of a chemical reaction, an electrochemical reaction, a biochemical reaction, a material conversion process, a physical processing process, a data conversion process, or a manufacturing process.

The instructions, when executed individually or collectively by the at least one processor, cause the electronic device to predict the contribution of each of the reaction factors and the yield with respect to a plurality of reaction factor combinations by differently setting the reaction factors, and determine a reaction factor according to a predefined criterion based on a predicted contribution and yield with respect to the plurality of reaction factor combinations.

Additional aspects of embodiments will be set forth in part in the description which follows and, in part, will be apparent from the description, or may be learned by practice of the disclosure.

### BRIEF DESCRIPTION OF THE DRAWINGS

The above and/or other aspects will be more apparent by describing certain embodiments with reference to the accompanying drawings, in which:

FIG. 1A is a diagram for illustrating a reaction, according to an embodiment;

FIG. 1B is a diagram for illustrating an explainable yield prediction model, according to an embodiment;

FIG. 2A is a diagram for illustrating a deep learning operation method using an artificial neural network (ANN);

FIG. 2B is a diagram for illustrating an explainable yield prediction system, according to an embodiment;

FIG. 3 is a diagram for illustrating a structure of an explainable yield prediction model, according to an embodiment;

FIG. 4 is a diagram illustrating an example of an explainable yield prediction model, according to an embodiment;

FIG. 5 is a diagram illustrating a result of predicting yield of a specific chemical reaction by using an explainable yield prediction model, according to an embodiment;

FIG. 6 is a diagram illustrating an example of a reaction, according to an embodiment;

FIG. 7 is a diagram illustrating an analysis result of the reaction of FIG. 6 using an explainable yield prediction model, according to an embodiment;

FIG. 8 is a flowchart illustrating an yield prediction model, according to an embodiment;

FIG. 9 is a flowchart illustrating a training method, according to an embodiment; and

FIG. 10 illustrates an electronic device according to an embodiment.

### DETAILED DESCRIPTION

The following structural or functional descriptions of examples disclosed in the present disclosure are merely intended for the purpose of describing the examples and the examples may be implemented in various forms. The examples are not meant to be limited, but it is intended that various modifications, equivalents, and alternatives are also covered within the scope of the claims.

Although terms of “first” or “second” are used to explain various components, the components are not limited to the terms. These terms should be used only to distinguish one component from another component. For example, a “first” component may be referred to as a “second” component, or similarly, the “second” component may be referred to as the “first” component within the scope of the right according to the concept of the present disclosure.

It should be noted that if it is described that one component is “connected”, “coupled”, or “joined” to another component, a third component may be “connected”, “coupled”, and “joined” between the first and second components, although the first component may be directly connected, coupled, or joined to the second component. On the contrary, it should be noted that if it is described that one component is “directly connected”, “directly coupled”, or “directly joined” to another component, a third component may be absent. Expressions describing a relationship between components, for example, “between”, “directly between”, or “directly neighboring”, etc., should be interpreted to be alike.

As used herein, the singular forms “a,” “an,” and “the” are intended to include the plural forms as well, unless the context clearly indicates otherwise. It should be further understood that the terms “comprises” and/or “comprising,” when used in this specification, specify the presence of stated features, integers, steps, operations, elements, components or a combination thereof, but do not preclude the presence or addition of one or more other features, integers, steps, operations, elements, components, and/or groups thereof.

In the present disclosure, the term “an embodiment” is intended to encompass one or more embodiments, rather than being limited to a single example. Furthermore, features described in embodiments may be combined and implemented together.

Unless otherwise defined, all terms, including technical and scientific terms, used herein have the same meaning as commonly understood by one of ordinary skill in the art to which the present disclosure pertains. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

The embodiments may be implemented as various types of products, such as, for example, a personal computer (PC), a laptop computer, a tablet computer, a smartphone, a television (TV), a smart home appliance, an intelligent vehicle, a kiosk, and a wearable device. Hereinafter, embodiments will be described in detail with reference to the accompanying drawings. In the drawings, like reference numerals are used for like elements.′

One or more embodiments of the present disclosure relate to process optimization in chemical and materials engineering, as well as machine learning (ML) and explainable artificial intelligence (XAI) applied to scientific experimentation. Specifically, the disclosed embodiments provide systems and methods for predicting and optimizing reaction or manufacturing process yields by analyzing the individual contributions of reaction factors using explainable ML models. These systems and methods are applicable across a range of domains, including chemical synthesis, biochemical reactions, semiconductor processing, and energy storage material development, where process variables and reaction factors critically affect the quality, efficiency, and yield of final products. The embodiments enable data-driven experimental optimization through explainable AI, facilitating a deeper understanding of the quantitative impact of each reaction factor, while also reducing experimental time, cost, and uncertainty in achieving high-yield outcomes across various industrial sectors.

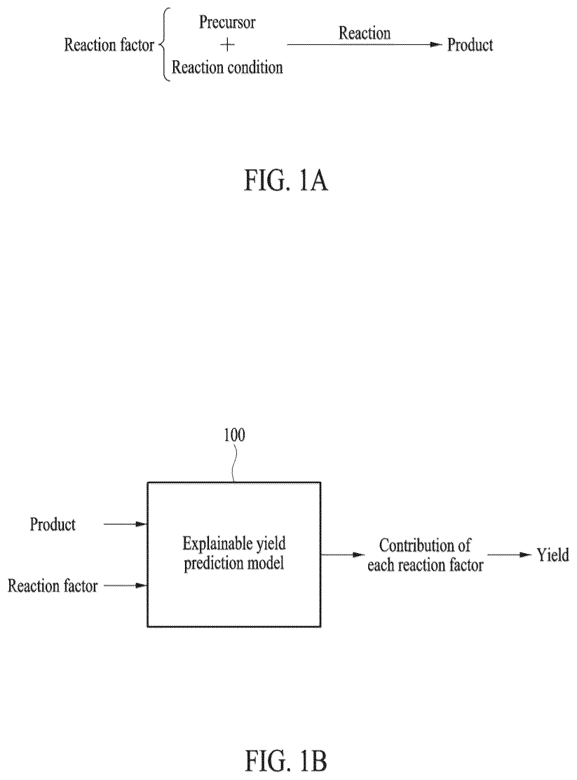

FIG. 1A is a diagram for illustrating a reaction, according to an embodiment.

Referring to FIG. 1A, a reaction according to an embodiment may refer to a process of forming a product as a specific input element changes. A reaction factor may be required to proceed with the reaction, and the reaction factor may include a precursor and a reaction condition. When the reaction is completed, the product may be generated.

The reaction in an embodiment may be performed under a specific condition, and the shape and composition of the product may vary depending on the characteristics and environment of the input element. The reaction in an embodiment may not be limited to a specific technical field and may be used in various application fields including material synthesis, semiconductor manufacturing, catalytic reaction, new pharmaceutical development, and energy storage device manufacturing. For example, in a chemical reaction, a raw material may be transformed along a specific reaction path and may become a final product. In a biochemical reaction, a precursor may be transformed into another compound due to the action of enzymes in a body, and in an electrochemical reaction, a precursor may be changed to a new chemical state by applying electrical energy to the precursor. In addition, in a physical transformation process, a composition of a material may change by physical manipulation, such as heating, compression, or melting.

The reaction factor in an embodiment may be an element that influences when the reaction is performed. The reaction factor may be referred to as a process variable, an input factor, or an input parameter. The reaction factor may be a factor that induces chemical, physical, or biological changes in a reaction process and may include the precursor and the reaction condition. The reaction factor may determine an environment in which a reaction occurs and may affect a result of the reaction. For example, in a catalytic reaction, a catalyst may act as the reaction factor and may adjust the reaction speed, and in the synthesis of battery electrode materials, a synthesis temperature or atmosphere gas may be the reaction factor. In a semiconductor process, a gas composition for specific impurity doping may act as the reaction factor, and in a biochemical reaction, a ligand that binds to a protein may be the reaction factor.

A product in an embodiment may be a final result generated by a reaction process, which occurs as a specific input element (e.g., precursor) undergoes transformation under defined reaction conditions. The characteristics of the product may vary depending on a reaction factor and a reaction condition, and the product may have different physical or chemical characteristics according to a specific technical field. For example, in chemical synthesis, the product may be a specific compound, and in a semiconductor process, a silicon thin film may be the product. In addition, in battery manufacturing, a synthesized electrode material may be the product, and in a biochemical reaction, a protein or a specific active material may be the product.

A precursor in an embodiment may be defined as a concept that includes all chemical elements participating in a reaction. The elements forming the reaction may be divided into a reactant, a catalyst, a ligand, a base, and a solvent. The reactant may be a main element that reacts to produce a final product. For example, in the Suzuki-Miyaura reaction or Buchwald-Hartwig reaction, aryl halide or boronic acid may be the reactants and a new product may be formed by combining those reactants. A catalyst may increase the reaction speed but may also act as a material that maintains its original state after the reaction. In organic synthesis, a palladium (Pd) catalyst and a nickel (Ni) catalyst may be typically used, and it may be important to analyze how high the yield of a specific catalyst is in a specific reaction.

A ligand may play a role in controlling a reaction by binding to a catalyst, and for example, in the Suzuki-Miyaura reaction, a phosphine ligand (PPh₃, SPhos, XPhos, etc.) may affect the reaction efficiency. In addition, a base may be used to maintain a state in which the reactivity is high in a specific chemical reaction, and a base, such as NaOH, K₂CO₃, and Cs₂CO₃, may vary depending on the reaction condition. Finding the most appropriate combination of a reactant and a base may be required to optimize a reaction. A solvent may play a role in dissolving a reactant for a smooth reaction, and in the Suzuki-Miyaura reaction, a solvent, such as THF/H₂O, MeCN/H₂O, and DMF/H₂O may be used. Hereinafter, all reaction elements, such as the reactant, the catalyst, the ligand, the base, and the solvent, may be referred to as precursors.

The reaction condition in an embodiment may be an environmental factor in which a reaction is performed and may be an important factor for determining the efficiency and selectivity of the reaction. The reaction condition may include not only a physical variable, such as temperature, pressure, and reaction time, but also a solvent environment and a characteristic of a device in which the reaction occurs. For example, the reaction temperature in a specific chemical reaction may affect not only the reaction speed but also the selectivity of a final product, and in a catalytic reaction, a specific solvent may affect the activity of the catalyst. In a semiconductor manufacturing process, plasma intensity or deposition speed may also act as reaction conditions, and in a biochemical reaction, the pH composition or concentration of a specific enzyme may be a reaction condition.

FIG. 1B is a diagram for illustrating an explainable yield prediction model, according to an embodiment. The description provided with reference to FIG. 1A may apply to FIG. 1B in substantially the same manner.

The yield may significantly vary depending on a reaction factor (e.g., a type of a precursor, a reaction temperature, etc.) while performing various reactions, and since the characteristic of a specific product varies for each reaction, it may be difficult to apply a general rule. Accordingly, repetitive experiments may be required to find an optimal reaction condition, and repetitively conducting experiments may be inefficient in terms of time and cost.

Various optimization algorithms have been proposed to optimize the reaction condition. However, most of the conventional methods focus on developing a model for predicting a reaction condition for a reaction factor and an input product or predicting yield under a specific condition. The model may provide a predicted value, but when the reaction is actually performed, a predicted result often may not match the experimental result. This may be a case in which the model is less reliable with respect to a product or reaction factor that is not learned, and in this case, the data may need to be complemented through an additional experiment or a reaction may need to be performed by using a new precursor.

An explainable yield prediction model 100 in an embodiment may quantitatively analyze the yield contribution of each reaction factor and may propose an experimental optimization direction in addition to a conventional simple yield prediction.

The explainable yield prediction model 100 may calculate the contribution of each reaction factor by receiving a product (e.g., a value or information representing a final resulting product that is generated through a reaction process) and a reaction factor as inputs. In one or more embodiments, the product and the reaction factor may be input into the explainable yield prediction model 100 either directly or indirectly. For example, the product and the reaction factor themselves may be provided as inputs, or values derived from them, such as pre-processed representations of the product and the reaction factor, may be used as inputs to the explainable yield prediction model 100. The contribution of each reaction factor may quantitatively show an impact of each reaction factor on the yield. The contribution of a specific reaction factor to the yield may be compared by analyzing the contribution of each reaction factor, and this may lead to the derivation of experimental design and optimization directions. While an yield prediction model in other embodiments may focus on predicting expected yield under a specific reaction condition, the explainable yield prediction model 100 in this embodiment may analyze the individual contribution of a reaction factor to help a researcher make more sophisticated decisions.

The contribution in an embodiment may refer to a value that may numerically express an impact of each reaction factor on the yield. A high contribution of a specific reaction factor may indicate that the reaction factor directly affects the yield and it is required to preferentially consider the reaction factor in the experimental optimization process. On the other hand, when the contribution of a specific reaction factor is low, the reaction factor may have little impact on the yield or may be highly affected by an interaction with another reaction factor.

For example, when comparing catalyst A with catalyst B in a specific reaction, if the contribution of catalyst A is 0.7 and the contribution of catalyst B is 0.2, catalyst A may have a greater impact on determining the yield of the reaction. In this case, the yield may be improved by adjusting the concentration of catalyst A or optimizing the reaction condition. In addition, whether a specific solvent in a specific temperature range maximizes the reaction efficiency may be determined by analyzing the reaction temperature and the contribution of the solvent.

The contribution of each reaction factor may be calculated by the explainable yield prediction model 100 trained based on experimental data and may dynamically vary depending on a reaction condition. Accordingly, the individual contribution for a specific product and a reaction combination may be assessed, and an assessment result may be reflected in the experimental design.

The yield in an embodiment may be the amount of generated product after performing a specific reaction and may be an important indicator to evaluate whether the reaction has been efficiently conducted. Typically, the yield may be measured based on the theoretical maximum production amount of a reaction precursor and may be expressed as a percentage (%) value by comparing the theoretical maximum product amount with the amount of the product obtained in the actual experiment.

For example, when a specific reaction may theoretically produce 100 g of products and 80 g of products is actually produced as a result of an experiment, the yield of the reaction may be calculated to be 80%. The yield may vary depending on the reaction factor and the reaction condition and may be maximized by setting an optimal experimental condition.

The yield may be an important indicator for evaluating the efficiency of a reaction, and achieving high yield may be the primary goal of reaction design and optimization. The explainable yield prediction model 100 may predict the yield based on an input reaction factor and may derive the experimental design and optimization directions by analyzing the contribution of each reaction factor.

The explainable yield prediction model 100 may provide the individual contribution of a reaction factor unlike an AI-based yield prediction model of a black box type. A researcher may improve the yield by using the information about the contribution of each reaction factor provided by the model and may design an experiment to optimize the reaction condition.

The explainable yield prediction model 100 may be used to reduce the number of experiments and set an optimal reaction condition. When the researcher searches for an optimal catalyst and an optimal reaction condition in a specific reaction, the explainable yield prediction model 100 may identify an element to be preferentially considered based on the contribution value of each reaction factor computed by the explainable yield prediction model 100. The researcher may maximize the yield by using the information and may reduce the experimental cost and time.

The explainable yield prediction model 100 in an embodiment may be used for not only a specific chemical reaction experiment but also various industrial fields. The researcher may optimize a combination of reaction factors by using the explainable yield prediction model 100, may reduce the experimental cost and time, and may design an experiment to improve the quality of a product. Specifically, the model may be applied to various industries, such as new pharmaceutical development, semiconductor and electronic material manufacturing, and battery material development.

According to an embodiment, the explainable yield prediction model 100 may be applied in the development of new pharmaceuticals. In a new pharmaceutical synthesis experiment, finding an optimal reaction condition may be significantly important, and the yield may significantly vary depending on a change in a reaction factor. The explainable yield prediction model 100 may predict an optimal combination of reaction factors based on the previous experimental data and by using this, thereby enabling efficient optimization of a synthesis process for a new pharmaceutical candidate material. For example, in a derivative synthesis experiment for a specific anticancer agent, the model may recommend an optimal combination of a catalyst and a solvent by learning from historical data. The researcher may reduce unnecessary repetitive experiments and save experimental cost and time by conducting the experiment by considering the reaction condition proposed by the model.

In addition, the explainable yield prediction model 100 may be used for a semiconductor and electronic material manufacturing process. In semiconductor manufacturing, various chemical reactions, such as thin film deposition, doping, and etching, may be performed and optimizing a reaction condition of each process may be significantly important. Specifically, in a chemical vapor deposition (CVD) process, conditions, such as a combination of reaction gases, temperature, and pressure, may determine the quality and property of a thin film. By using the explainable yield prediction model 100, the optimal thickness and uniformity of the thin film may be ensured by adjusting specific precursor gases and reaction conditions. For example, while forming a thin film on a silicon wafer, the model may learn the previous data to recommend an optimal combination of precursors and a reaction condition. The researcher may optimize a process condition by using the recommended information and may improve the performance and yield of a semiconductor device.

According to an embodiment, the explainable yield prediction model 100 may be applied to battery (energy storage) material development. The performance of a lithium-ion battery may significantly rely on the characteristics of cathode and anode materials, and a combination of precursors and a reaction condition may act as important variables in the process of synthesizing the materials. By using the explainable yield prediction model 100, a combination for inducing the optimal battery performance may be found by adjusting a specific precursor (e.g., a ratio combination of nickel, cobalt, and manganese) and a reaction condition. For example, when manufacturing a high-nickel cathode material, the model may recommend an optimal reaction temperature and a solvent combination to optimize the experiment to increase the energy density. The researcher may effectively adjust the experimental condition based on the yield predicted by the model and an analysis result of the contribution of each reaction factor, and may improve the performance of the battery material.

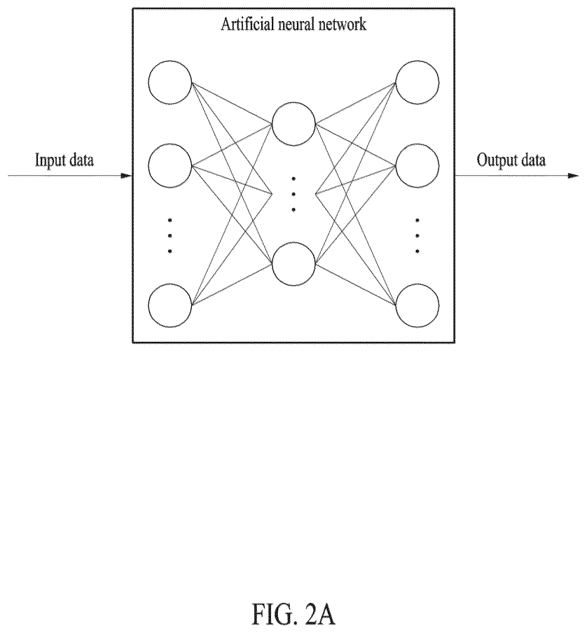

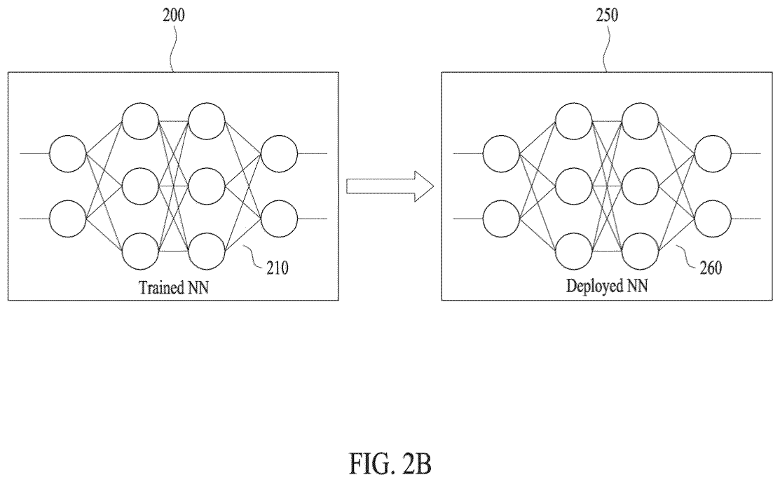

A deep learning operation method using an artificial neural network (ANN) is described with reference to FIGS. 2A and 2B before describing detailed operations of the explainable yield prediction model 100 in an embodiment.

FIGS. 2A and 2B are diagrams for illustrating a deep learning operation method using an ANN.

An artificial intelligence (AI) algorithm including deep learning may input the input data to an ANN, may learn output data through an operation such as convolution, and may extract a feature by using the trained ANN. The ANN may refer to a computational architecture that models a biological brain. In the ANN, nodes corresponding to neurons of the brain may be connected to each other and collectively operate to process input data. Various types of neural networks may include, for example, a convolutional neural network (CNN), a recurrent neural network (RNN), a deep belief network (DBN), or a restricted Boltzmann machine (RBM) but are not limited thereto. In a feed-forward neural network, neurons may have links to other neurons. The links may be expanded in a single direction, for example, a forward direction, through a neural network.

FIG. 2A illustrates a structure of an ANN (e.g., a CNN) that receives input data and outputs output data. The ANN may be a deep neural network including two or more layers.

FIG. 2B is a diagram for illustrating an explainable yield prediction system, according to an embodiment.

Referring to FIG. 2B, an explainable yield prediction system in an embodiment may include a training device 200 and an inference device 250. The training device 200 in an embodiment may be a computing device having various processing functions, such as functions for generating a neural network, training (or learning) the neural network, and retraining the neural network. For example, the training device 200 may be implemented as various types of devices, such as a PC, a server device, and a mobile device.

The training device 200 in an embodiment may generate one or more trained neural networks 210 by iteratively training (or learning) a given initial neural network. Generating the one or more trained neural networks 210 may be determining a parameter of the neural network. In this case, the parameter may include various types of data that is input to or is output from a neural network, such as I/O activations, weights, and biases of the neural network. As the neural network is iteratively trained, the parameters of the neural network may be tuned to compute a more accurate output for a given input.

The training device 200 in an embodiment may transmit the one or more trained neural networks 210 to the inference device 250. The inference device 250 may be included in a mobile device, an embedded device, and the like. The inference device 250 in an embodiment may be dedicated hardware for driving the neural network and may be an electronic device including at least one of a processor, a memory, an input/output (I/O) interface, a display, a communication interface, or a sensor.

The inference device 250 in an embodiment may include a digital device with computational capabilities, incorporating memory and a microprocessor. Examples of the inference device 250 may include a personal computer (PC) such as a laptop, a tablet PC, an AI speaker, a smart TV, a mobile phone, a navigator, a web pad, a personal digital assistant (PDA), and a workstation.

The inference device 250 in an embodiment may drive the one or more trained neural networks 210 or may drive a neural network 260 in which the one or more trained neural networks 210 are processed (e.g., quantized). The inference device 250 for driving the processed neural network 260 may be implemented in an independent device that is separated from the training device 200. However, the example is not limited thereto, and the inference device 250 may be implemented in the same device as the training device 200.

In one or more embodiments, the deployed neural network 250 may represent a fully trained neural network that has completed its initial training phase. Upon deployment, the neural network 250 may enter an inference phase, during which it performs prediction tasks based on input data. However, in certain embodiments, the deployed neural network 250 may continue to undergo additional training during or after deployment. This enables continuous learning or adaptive refinement of the model while concurrently performing inference tasks.

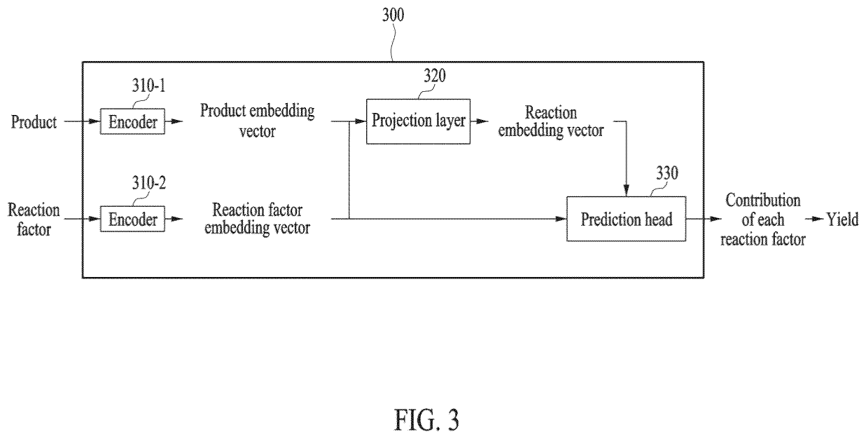

FIG. 3 is a diagram for illustrating a structure of an explainable yield prediction model, according to an embodiment. The description provided with reference to FIGS. 1A to 2B may substantially equally apply to FIG. 3.

Referring to FIG. 3, an explainable yield prediction model 300 in an embodiment may receive a product and a reaction factor as inputs, may generate a product embedding vector and a reaction factor embedding vector, and may predict final yield by computing the contribution of each reaction factor based thereon.

The explainable yield prediction model 300 may generate embedding vectors by inputting the product and the reaction factor to encoders 310-1 and 310-2, respectively. In one or more embodiments, two separate encoders 310-1 and 310-2 may be used to process the product and the reaction factor, respectively. The encoders 310-1 and 310-2 may have identical or different neural network architectures. In other embodiments, although FIG. 3 illustrates the use of two separate encoders 310-1 and 310-2, a single integrated encoder may be used to process both the product and the reaction factor, either in parallel or in a sequential manner. The explainable yield prediction model 300 may generate a product embedding vector and a reaction factor embedding vector by individually encoding the product and the reaction factor, respectively, and may analyze a relation between the reaction factor and the product by using the embedding vectors. The encoders 310-1 and 310-2 may be used to transform raw input data (like molecules, materials, and process parameters) into machine-understandable representations (embeddings). The encoders 310-1 and 310-2 may include an input layer to receive raw data, an embedding layer to convert raw data to dense vectors, a feature extraction layer configured to extract features relevant to a prediction task, a normalization layer, an activation function layer, and an output layer to output embedding vectors. For example, the product may be input to the encoder 310-1 and may be converted into the product embedding vector, and the reaction factor may be input to the encoder 310-2 and may be converted into the reaction factor embedding vector.

The reaction factor embedding vector may be a multidimensional vector that reflects the characteristic of the reaction factor and may indicate how each response factor acts in a reaction process. The reaction factor may include a precursor and a reaction condition and may affect the formation and yield of the product in the reaction process.

The reaction factor embedding vector may be generated as a result of individually encoding each reaction factor. For example, when the reaction factor is a catalyst, information, such as an active part, reaction affinity, and concentration, may be included in the reaction factor embedding vector. Properties, such as polarity, a dielectric constant, and viscosity may be reflected in a solvent. A reaction condition, such as temperature and pressure, may also be included in the reaction factor embedding vector, and reaction activation energy in a specific temperature range or an equilibrium state may be used as embedding information.

The product embedding vector may be a multidimensional vector that reflects the characteristic of the product and may be used as information for determining how the explainable yield prediction model 300 considers the product in the reaction process. The product may refer to a final product produced by a specific reaction, and the product embedding vector may include information, such as the structural characteristic and reactivity of the product.

For example, when the product is an organic compound in a specific chemical reaction, the product embedding vector may include a feature, such as a molecular structure, a functional group, and an electron distribution of the compound. In a semiconductor process, when the product is a specific thin film material, the product embedding vector may include a composition, a crystal structure, and the electrical characteristic of the thin film. When the product is a specific electrode material in an electrochemical reaction, the product embedding vector may include an element, such as a surface area of an electrode, conductivity, and catalytic activity.

A reaction embedding vector in an embodiment may be an embedding vector for the entire reaction and may be generated by aggregating the product embedding vector and the reaction factor embedding vector. The reaction embedding vector may be generated via a projection layer 320 and may be input to a prediction head 330 together with the reaction factor embedding vector. The projection layer 320 may refer to a neural network layer configured to transform one or more input vectors into a different vector space (e.g., of a different dimension) to make the data suitable for a specific task. For example, the projection layer 320 may include a fully connected layer that performs a learnable linear transformation, optionally followed by a non-linear activation function.

The reaction embedding vector may also play a role in representing the entire reaction in a single vector space by aggregating characteristics of individual reaction factors. The individual reaction factors may independently act in each reaction process but may also interact with each other according to a specific combination. The explainable yield prediction model 300 may reflect the characteristic of the entire reaction considering the interaction between the reaction factors by generating the reaction embedding vector.

For example, when a reaction factor includes a catalyst and a solvent in a specific reaction, the individual reaction factor embedding vectors may respectively reflect the characteristics of the catalyst and the solvent, but the reaction embedding vector may indicate the characteristic of the entire reaction by reflecting the interaction between the catalyst and the solvent. When a specific catalyst increases or inhibits reaction activity in a specific solvent environment, the reaction embedding vector may learn this relation.

The prediction head 330 in an embodiment may compute the contribution of each reaction factor and may predict the final yield based on the contribution of each reaction factor. The contribution of each reaction factor may quantitatively represent an impact of a specific reaction factor on the yield and may be used for reaction optimization and experimental design.

After the explainable yield prediction model 300 converts information about an input product and a reaction factor into a vector, the explainable yield prediction model 300 may predict the final yield by analyzing the contribution of each reaction factor.

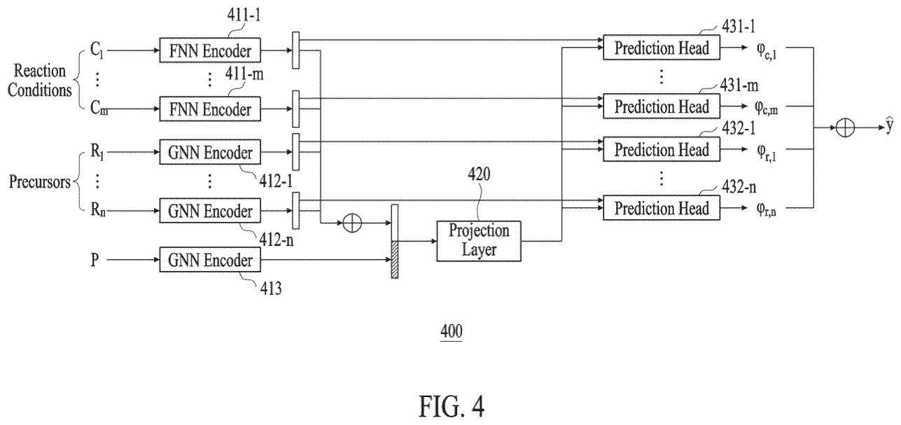

FIG. 4 is a diagram illustrating an example of an explainable yield prediction model, according to an embodiment. The description provided with reference to FIGS. 1A, 1B, 2A, 2B, and 3 may apply to FIG. 4 in substantially the same manner.

An explainable yield prediction model 400 in an embodiment may receive a product (P) and a reaction factor including a reaction condition (C₁, C₂, . . . , Cₘ) and a precursor (R₁, R₂, . . . , Rₙ) as inputs, may generate embedding vectors by respective encoders, may predict a contribution of each reaction factor by using the embedding vectors, and may predict yield of the entire reaction.

The explainable yield prediction model 400 may input the reaction condition to fully connected neural network (FNN) encoders 411-1 to 411-m and may input the precursor to graph neural network (GNN) encoders 412-1 to 412-n and may generate respective embedding vectors. Since the reaction condition and the precursor have inherently different properties, different types of encoders may be used to effectively reflect their characteristics.

The reaction condition in an embodiment may be converted into a reaction condition embedding vector by the FNN encoders 411-1 to 411-m. The reaction condition may include an environmental element in which a specific reaction is performed and may include numerical or categorical data, such as temperature, pressure, a solvent, and a reaction time. The reaction condition embedding vector may be a vector that numerically represents a condition in which the specific reaction is performed. For example, the temperature, pressure, reaction time, and characteristic of the solvent (viscosity, polarity, etc.) may be included in the reaction condition embedding vector. For example, when a specific chemical reaction is performed under conditions of temperature 80° C. and pressure 2 atm, the conditions may be represented as multidimensional vectors having the same size through the FNN encoders 411-1 to 411-m.

The FNN encoders 411-1 to 411-m in an embodiment may generate an embedding in the multidimensional vector format by receiving the reaction condition. For example, when a temperature is 100° C., pressure is 1 atm, and a solvent is methanol in a specific reaction, the FNN encoder may quantify and convert the information into an embedding vector having a fixed size.

The precursor in an embodiment may be converted into a precursor embedding vector by the GNN encoders 412-1 to 412-n. The precursor may have, for example, a molecular structure of an organic compound. The molecular structure of the precursor may include complex characteristics, such as an interatomic bonding relationship, a stereostructure, and isomeric information, and a GNN encoder may be used to effectively represent the information.

The GNN encoders 412-1 to 412-n in an embodiment may represent the precursor as a graph and may receive the precursor. For example, in the case of an organic compound, an atom may be modeled as a node of a graph and a bond between atoms may be modeled as an edge of the graph. The GNN encoder may generate an embedding vector that reflects an electron distribution, reactivity, and a steric effect of the molecule by learning the graph structure.

An approach of simply converting the precursor into an image and inputting the image to the FNN encoder may also be considered, but this approach may have a limitation in fully reflecting an intrinsic chemical structure of the precursor. On the other hand, the GNN encoder may effectively learn the information including the interatomic bonding relationship, the stereostructure, and electron characteristics by using the characteristics of the graph data and may thus be advantageous in accurately representing the precursor and improving the accuracy of yield prediction.

More specifically, the precursor may typically have a chemical structure formed by bonds with atoms and when representing the structure as a graph, the atom may be modeled as a node of the graph and the bond between atoms may be modeled as an edge of the graph. For example, in an aromatic compound, such as benzene (C₆H₆), six carbon atoms may be bonded in a ring shape and single and double bonds may form a resonance structure. This structure may be an important factor to determine the distribution and reactivity of electrons, and a graph-based model may be required to learn the structure. This chemical relationship may be naturally reflected by using the GNN encoder, and the extracted features may include physiochemical properties, such as bond strength between atoms, polarity, and electron density.

On the other hand, converting the precursor into an image and inputting the image to the FNN encoder may not clearly reflect the structural relationship of the precursor. In an image-based input scheme, a chemical bond and an atomic arrangement may be converted pixel-wise and consequentially, the chemical meaning may not be directly reflected. For example, when a molecule including a benzene ring has a similar pixel pattern to a molecule that does not include the benzene ring, the FNN encoder may not effectively distinguish chemical differences between the two molecules. Additionally, stereochemical information of the molecule, in other words, spatial characteristics, such as cis/trans isomerism or chirality, may be distorted. Since the structural information may significantly affect the chemical reactivity, the graph-based model may be more appropriate to accurately reflect it.

Additionally, the physiochemical properties of the precursor may be included as node and edge properties by using the GNN encoder. For example, information, such as charge, polarity, solubility, and reactivity of atoms may be added as the node and edge properties of the graph. These physicochemical properties may be important elements affecting the reactivity and reaction speed of the precursor, and these properties may not be directly reflected by the FNN encoder.

The GNN encoder may also be effective in learning a substructure of a molecule. Typically, a molecule may include various functional groups and the functional groups may significantly affect the reactivity and yield of the molecule. For example, a specific functional group, such as a benzene ring (aromatic ring), an amine group (—NH₂), and a carbonyl group (—C═O), may show specific reactivity. The GNN encoder may learn a pattern of the substructure in the graph structure and may predict the reactivity by detecting a similar pattern in a new molecule. On the other hand, in the image-based approach, the substructure may be converted into a single image and may be processed, and thus, it may be difficult to learn which part is important.

A vector representation scheme, such as molecular fingerprinting or one-hot encoding, may need to be used to input a precursor to the FNN encoder. This scheme may allow the FNN to process the precursor by converting the precursor into a vector in a fixed dimension but may not directly reflect the structural relationship between atoms. For example, a molecule including an aromatic ring and a molecule that does not include an aromatic ring may have a similar vector representation, and this may degrade the prediction accuracy of the model. In addition, as the size of the molecule increases, the vector dimension may increase, and this may cause an overfitting problem in the learning process.

The FNN encoders 411-1 to 411-m and the GNN encoders 412-1 to 412-n in an embodiment may be set to output embedding vectors having the same size. Since the reaction condition embedding vector and the precursor embedding vector are represented in a vector space in the same dimension, the two vectors may be processed in the same operation structure, and a vector operation may be performed while learning an interaction.

Since the reaction condition embedding vector and the precursor embedding vector may be normalized in a multidimensional vector having the same size of each property, the explainable yield prediction model 400 may perform an operation between the two vectors. The explainable yield prediction model 400 may generate a single summed embedding vector by performing a summation operation (i.e., symbol @ shown in FIG. 4) on the precursor embedding vectors and the reaction condition embedding vectors. The summed embedding vector may be concatenated with a product embedding vector, wherein the explainable yield prediction model 400 may input the product P to a GNN encoder 413 to obtain the product embedding vector. A concatenation operation may refer to a process of forming a new embedding vector by concatenating or joining two or more individual embedding vectors end-to-end along a specific dimension, and the new embedding vector may be input to a projection layer 420 as a reaction embedding vector that represents the entire reaction. In one or more embodiments, the new embedding vector may be input into the projection layer 420 either directly or indirectly. For example, the new embedding vector itself may be provided as an input, or a value derived from it, such as a pre-processed representation of the new embedding vector, may be used as an input to the projection layer 420. The projection layer 420 may reduce or convert a dimension of a concatenated dimension for ease of the following operation.

The reaction embedding vector generated as described above may be a vector representation that comprehensively reflects the characteristics of the entire reaction and may contain the information about the entire reaction. For example, when a specific chemical reaction is performed under a specific precursor combination and a specific reaction condition, the reaction embedding vector may indicate a reaction condition with the precursor and an interaction between products.

The reaction embedding vector may be input to prediction heads 431-1 to 431-m and 432-1 to 432-n corresponding to respective reaction factor embedding vectors. For example, an embedding vector of temperature may be input to the prediction head 431-1 corresponding to the temperature together with the reaction embedding vector and the contribution φ_{c,1} of the temperature may be calculated, and an embedding vector of a solvent may be input to the prediction head 431-m together with the reaction embedding vector and the contribution of the solvent may be calculated. Similarly, embedding vectors of precursors may be input to the precursor prediction heads 432-1 to 432-n together with the reaction embedding vector, respectively, and the contributions of respective precursors may be calculated. For example, when an embedding vector of aryl halide is input to the precursor prediction head 432-1 together with the reaction embedding vector, the precursor prediction head 432-1 may quantitatively calculate the contribution of aryl halide to the yield.

The contributions of respective reaction factors φ_{c,1}, . . . , φ_{r,n} calculated by the prediction heads 431-1 to 431-m and 432-1 to 432-n may be summed and predicted yield ŷ of the entire reaction may be calculated. For example, when there are three reaction factors, a reaction time (a reaction condition), a solvent (a reaction condition), and a substrate material (a precursor) in a semiconductor manufacturing process, the contribution φ_{c,1} of the reaction time, the contribution φ_{c,2} of the solvent, and the contribution φ_{r,1} of the substrate material may be calculated to be 0.05, 0.05, and 0.7 by the prediction heads corresponding to the reaction factors, respectively, and accordingly, the predicted yield ŷ may be calculated to be 0.05+0.05+0.7=0.8.

The prediction heads 431-1 to 431-m and 432-1 to 432-n may quantitatively and independently compute an impact of each reaction factor on the yield and may accurately compute the predicted yield y of the entire reaction by summing the contributions thereof. The researcher may easily identify a positive or negative impact of each reaction factor on the result through the computed predicted yield and may optimize the experimental condition or reaction factor to reach desired target yield based on the identified impact. The researcher may refer to an entity that designs, experiments, analyzes, and optimizes a reaction. The researcher may analyze the contribution of the reaction factor by using the explainable yield prediction model 400, may search for an optimal reaction condition, and may design the experiment. Additionally, the researcher may review a combination of reaction factors recommended by the explainable yield prediction model 400 and may continuously train the model by generating new experimental data as needed. The researcher may be referred to as a user, an engineer, a scientist, or an experimenter.

A training device (e.g., the training device 200 described with reference to FIG. 2B) may train the explainable yield prediction model 400 by optimizing a weight of a neural network in the prediction heads 431-1 to 432-n, the projection layer 420, and the encoders 411-1 to 412-n and 413 of the prediction model to minimize the objective function by using training data.

The training device 200 may receive input data and ground truth data from the training data to train the explainable yield prediction model 400 and may update a parameter of the model to minimize a given objective function. The training device 200 may generate a model for accurately predicting the yield from the input data and explaining the contribution of each reaction factor by adjusting a parameter (e.g., a weight) of the neural network model to minimize a value of the objective function.

The training device 200 may include a term that minimizes a difference between a predicted default yield value and a yield value of the ground truth data as the objective function. In other words, for a given reaction factor R and a product P, a parameter of the model may be optimized so that the yield ŷ computed by the explainable yield prediction model 400 becomes as close as possible to ground truth yield y included in the training data.

The training device 200 may additionally use auxiliary objective functions for the contribution of each reaction factor so that the model has an explainable property. Specifically, the training device 200 may set the objective function to balance the contribution of each reaction factor for each specific factor type (e.g., a catalyst, a solvent, and a base). The training device 200 may adjust the contributions of reaction factors belonging to the same category to set an average impact on the yield in the specific factor type to be close to “0”. This may allow the researcher to clearly analyze which individual factor has a positive or negative impact on the yield for each factor type.

Additionally, the training device 200 may additionally include a term for a variance of the contribution of each factor in the objective function. For a factor that does not substantially affect the yield among specific reaction conditions or precursors, the objective function may be set to correct the contribution value to be close to “0”. For example, among temperature, pressure, and a solvent, when a change in temperature has little impact on the yield in a specific reaction, the contribution of temperature may be trained to be close to “0”. As described above, a normalization term may be added to the objective function to minimize a deviation of contributions and allow the yield prediction model to automatically filter an unnecessary variable in the reaction.

The training device 200 may train the explainable yield prediction model 400 by considering all of the objective function terms described above and optimizing the objective function terms. The training device 200 may optimize the model by considering not only the improvement of fundamental yield prediction accuracy but also the balance of average contribution of each factor type and the effect of reducing the contribution of an unnecessary reaction factor. The objective function used by the training device 200 may be expressed by Equation 1 below.

$$\tilde{J} = J + α \cdot \frac{1}{m} {\sum }_{j = 1}^{m} {( \frac{1}{N} {\sum }_{i = 1}^{N} {∅}_{i}^{j} )}^{2} + β \cdot \frac{1}{N} {\sum }_{i = 1}^{N} ( \frac{{\sum }_{j = 1}^{m} {( {∅}_{i}^{j} )}^{2} / m}{{\sum }_{j = 1}^{m} {( {∅}_{i}^{j} - {\overline{∅}}^{i} )}^{2} / ( m - 1 )} ) \qquad [ Equation 1 ]$$

In Equation 1, J may be a term that denotes the difference between predicted yield and ground truth yield and may be expressed by Equation 2 below.

$$𝒯 = ( 1 - λ ) \cdot \frac{1}{N} {\sum }_{i = 1}^{N} {( {y}_{i} - {\hat{μ}}_{i} )}^{2} + λ \cdot \frac{1}{N} {\sum }_{i = 1}^{N} [ \frac{{( {y}_{i} - {\hat{μ}}_{i} )}^{2}}{{\hat{σ}}_{i}^{2}} + log {\hat{σ}}_{i}^{2} ] \qquad [ Equation 2 ]$$

In Equation 2, \hat{μ}ᵢ may denote a yield prediction value of the model, and

$${\hat{σ}}_{i}^{2}$$

may denote a value indicating uncertainty (variance) of the prediction computed by the model. As the uncertainty decreases, the model may perform a more reliable prediction.

In Equation 1, the second term may be a term that causes an average of contributions of reaction factor types to approach 0 and this may adjust the model to prevent from being biased to a reaction factor of a specific type. α may be a hyperparameter indicating a relative weight of the objective function, and as the value increases, the average of contributions for factor types may be strongly adjusted to 0. The value may be experimentally set and may be, for example, set to 5. m may be the number of total reaction factors, and N may be the number of samples in the training data and may refer to the number of total reaction cases included in the training data.

$${∅}_{j}^{i}$$

may denote the contribution of a j-th reaction factor to the yield in an i-th reaction case in the training data.

In Equation 1, the third term may be a term for correcting the contribution based on the variance of reaction factor contributions and may adjust the contribution of a reaction factor that does not contribute to the yield to converge closer to 0. β may be a hyperparameter for a term that controls the deviation of contributions, and as the value increases, the contribution value of a factor that is not important in the reaction may be strongly reduced. For example, a small value, such as 0.05, may be selected experimentally. Ø̅ⁱ may denote an average of all reaction factor contributions in a specific reaction case i. A value

$${\sum }_{j = 1}^{m} {( {∅}_{j}^{i} )}^{2} / m$$

in the numerator may represent a square mean of contribution of factors in the specific reaction case i and may be used as an indicator for representing the absolute strength of the contribution. A value

$${\sum }_{j = 1}^{m} {( {∅}_{j}^{i} - {\overline{∅}}^{i} )}^{2} / ( m - 1 )$$

in the denominator may represent a contribution variance of each factor in the specific reaction case i and may be used as an indicator for representing how clearly the contributions of respective factors are distinguished from each other.

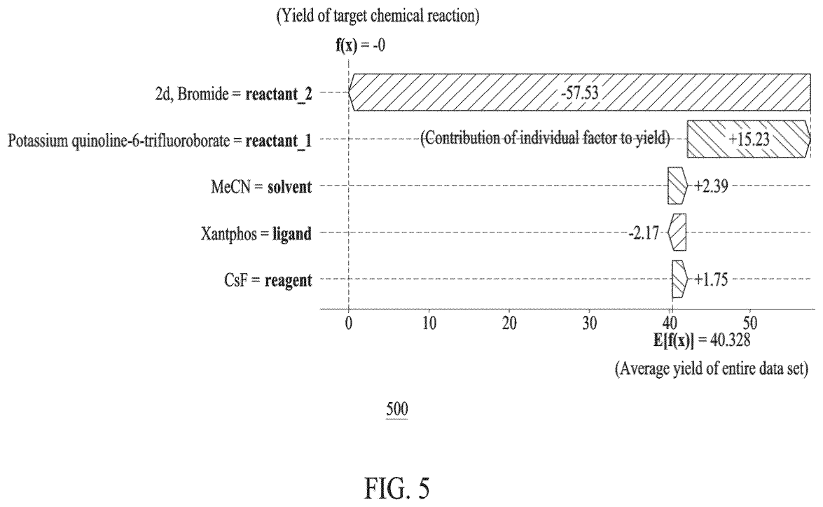

FIG. 5 is a diagram illustrating a result of predicting yield of a specific chemical reaction by using an explainable yield prediction model, according to an embodiment. The description provided with reference to FIGS. 1A, 1B, 2A, 2B, 3, and 4 may substantially equally apply to FIG. 5.

Referring to FIG. 5, the horizontal axis of a graph 500 in an embodiment may indicate yield and the vertical axis may indicate an individual reaction factor used for yield prediction. A reference line shown in the central portion of the graph 500 may indicate average yield of chemical reactions included in the entire training data set. The average yield value may be an average of yield values obtained from the entire data set that the model learns, and in the example of FIG. 5, the value may be about 40.3. The average value of yields may be used as a criterion to determine whether the yield of a specific reaction is high or low.

An explainable yield prediction model may independently predict the contribution of each reaction factor to the entire reaction yield. For example, in the graph 500, a second reactant reactant_2 2d.Bromide, which is a reaction factor, may have a contribution of −57.53, and this may indicate that the second reactant has a significantly adverse impact on the yield. In other words, a reaction including reactant_2 may have significantly low yield compared to the average yield value. On the other hand, a first reactant reactant_1 potassium quinoline-6-trifluoroborate, which is another reactant, may have a contribution of 15.23, and this may indicate that the first reactant positively contributes to the improvement of the yield.

A solvent MeCN may have a contribution of +2.39, and this may indicate that the solvent has a slightly positive impact compared to the average yield. A ligand Xantphos may have a contribution of −2.17, and this may indicate that the ligand has an adverse impact on the yield. In addition, a reagent CsF may have a contribution of +1.75, and this value may indicate that the reagent has a slightly positive contribution to the yield.

The explainable yield prediction model may quantitatively provide the contributions of individual reaction factors to the yield as the graph 500 and may clearly provide a positive or negative contribution level of each reaction factor. The researcher may intuitively identify an impact of a specific reaction condition or precursor on a reaction result through the contribution and may perform more efficient experimental design and optimization. For example, the researcher may conduct an experiment by replacing a precursor having a significantly low contribution in the yield prediction result with another material or changing a ligand that is predicted to have an adverse impact to another ligand to increase the yield of the experiment. Conversely, the researcher may additionally increase the yield of the experiment by performing the reaction design based on a reaction condition having a significantly positive contribution.

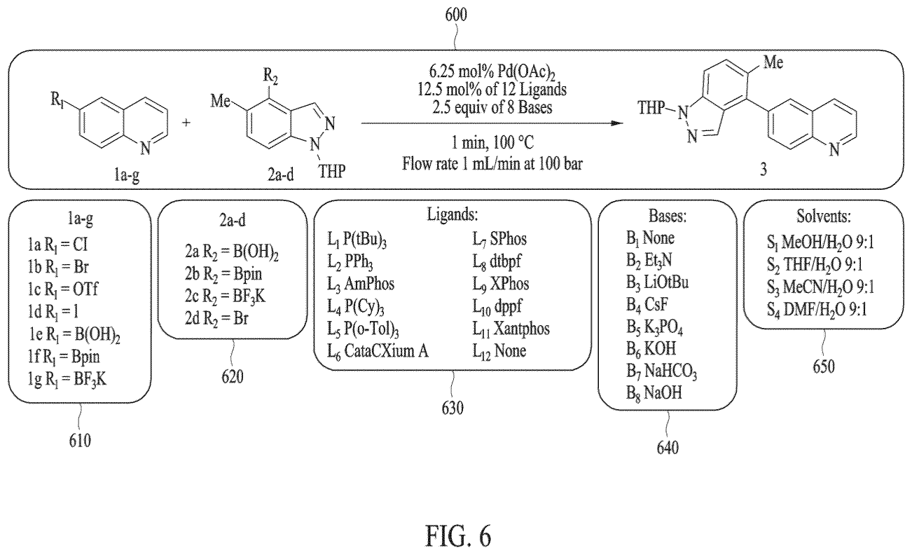

FIG. 6 is a diagram illustrating an example of a reaction, according to an embodiment. The description provided with reference to FIGS. 1A, 1B, 2A, 2B, and 3 to 5 may apply to FIG. 6 in substantially the same manner.

Referring to FIG. 6, in a reaction 600 in an embodiment, a product may be an organic compound including a quinoline scaffold and may be formed by combining two different types of reactants.

Reactants used for producing the product may be divided into two categories, a first reactant 1a-g 610 and a second reactant 2a-d 620. The first reactant 1a-g 610 may have a quinoline structure in which a substituent is variously substituted with Cl, Br, OTf, I, B(OH)₂, Bpin, and BF₃K, and the second reactant 2a-d 620 may include a compound in which a substituent is substituted with B(OH)₂, Bpin, BF₃K, and Br. A reaction may be performed by selecting respective candidates from two categories of reactants. For example, the reaction 600 may be performed by selecting 1a(R₁=Cl) from the first reactant 610 and 2a(R₂=B(OH)₂) from the second reactant 620.

When performing the reaction 600, the yield of the product may be affected by reaction conditions, such as a ligand, a base, and a solvent. A ligand 630 may be a chemical material that adjusts the activity or selectivity of the reaction by bonding to a catalyst in a chemical reaction and may be provided as 12 candidates including, for example, P(tBu)₃, XPhos, Xantphos, and PPh₃fmf. A base 640 may be a material that may contribute to stimulate the reactivity of the chemical reaction or create a neutralizing environment, and there may be eight base candidates including NaOH, K₃PO₄, CsF, and LiOtBu. A solvent 650 may be a medium that may adjust the reactivity by providing an environment in which the reaction is performed, and four candidates may be provided.

In the reaction 600, the reaction may be performed by selecting one candidate from each of reaction factors of each category, and the number of all possible combinations may be seven from the first reactant 610, four from the second reactant 620, 12 from the ligand 630, eight from the base 640, and four from the solvent 650, and thus, the number of combinations thereof may be 7×4×12×8×4=10,752.

A total of 10,752 experiments may be conducted to find the best reaction factor combination to obtain the product. However, when using the explainable yield prediction model in an embodiment, the researcher may immediately predict the yield of the reaction combination without actually conducting individual experiments if information about the product and the reaction factor is provided.

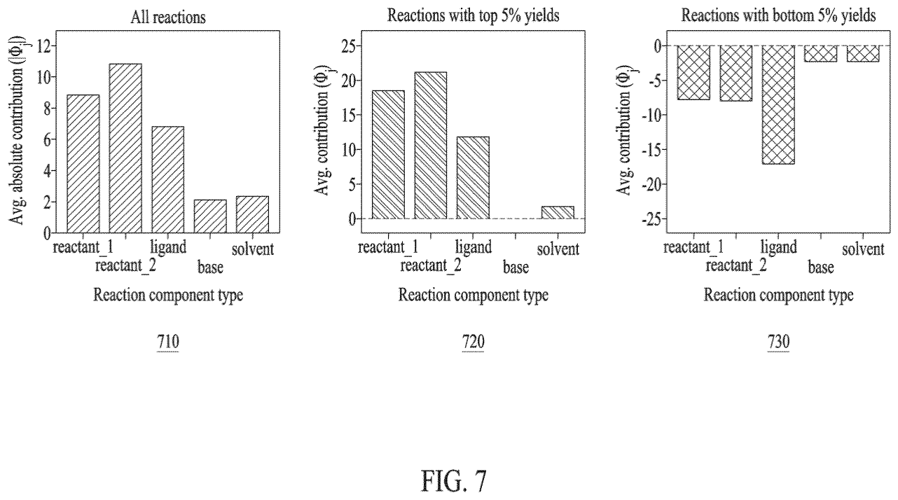

FIG. 7 is a diagram illustrating an analysis result of the reaction of FIG. 6 using an explainable yield prediction model, according to an embodiment. The description provided with reference to FIGS. 1A, 1B, 2A, 2B, and 3 to 6 may apply to FIG. 7 in substantially the same manner.

Referring to FIG. 7, a first graph 710 may represent an average absolute contribution of each reaction factor to the yield with respect to all reactions shown in FIG. 6. In this case, the average absolute contribution may denote an average value of absolute values of degrees of positive or negative impacts of reaction factors on the yield. For example, the first reactant reactant_1 may have an absolute contribution of about 9 and the second reactant reactant_2 may have the greatest absolute contribution of about 11. On the other hand, the absolute contributions of a ligand, a base, and a solvent may be relatively low, and accordingly, it may suggest that the first reactant reactant_1 and the second reactant reactant_2 have the greatest impacts on a yield change.

A second graph 720 may represent an average degree of positive contributions of reaction factors with respect to cases with top 5% yields among all reactions, in other words, reaction cases with the highest yields. The first reactant reactant_1 and the second reactant reactant_2 may have high positive contributions of about 20, respectively, and specifically, it may be suggested that the second reactant reactant_2 may act as an important factor in a reaction with high yield because the contribution of the second reactant reactant_2 is the greatest. The ligand may have a moderate positive contribution of about 10, and the solvent may have a lower contribution than the ligand. The base may have a relatively slight positive contribution in the graph 720.

Conversely, a third graph 730 may represent an average degree of negative contributions of reaction factors in cases with bottom 5% yields among all reactions, in other words, reaction cases with the lowest yields. Specifically, the ligand may have a contribution of about −18 and may be a reaction factor that has the greatest impact on the yield reduction. This may indicate that inappropriate selection of the ligand may significantly affect the yield reduction. The first reactant reactant_1 and the second reactant reactant_2 may have negative contributions of about −7, and the base and the solvent may have relatively low negative contributions.

The explainable yield prediction model in an embodiment may recommend an optimal reaction combination based on an analysis result in addition to simply providing the contribution of each reaction factor. After the yield prediction model quantitatively assesses the contribution of each reaction factor, the yield prediction model may optimize the combination of reaction factors to help the researcher predict an optimal experimental condition before conducting the experiment.

The explainable yield prediction model may analyze the contribution of each reaction factor first and may distinguish a factor that has a positive impact on the yield from a factor that has a negative impact on the yield. For example, when a ligand has the greatest impact on the yield reduction in a specific reaction, the model may propose the researcher to replace the ligand. On the other hand, in a reaction with high yield, a specific reaction factor (e.g., a specific reactant or a specific ligand) may make a significant contribution, and the model may preferentially consider a combination including the reaction factor.

To find an optimal combination of reaction factors, the explainable yield prediction model may apply an optimization algorithm to find a condition for maximizing the yield. For example, the explainable yield prediction model may efficiently find a combination with a high probability of high yield based on the accumulated data to date by applying Bayesian optimization. In addition, the explainable yield prediction model may preferentially select a combination with high yield and may iteratively assess the combination by considering various reaction factors as individual entities by using a genetic algorithm. The explainable yield prediction model may find an optimal combination by gradually modifying a reaction combination to increase the yield based on the contribution of each reaction factor by using gradient-based optimization.

The explainable yield prediction model may generate the optimal reaction combination through the optimization process and may recommend the optimal reaction combination to the researcher. The researcher may conduct the experiment by referring to the reaction factor combination recommended by the model and may set a condition that requires to include or exclude a specific reaction factor. When this condition is applied, the model may find the optimal combination of reaction factors by reflecting a constraint designated by the researcher.

The explainable yield prediction model may continuously learn an experimental result conducted by the researcher and may provide more precise recommendation. After the researcher experiments with the recommended combination and inputs the data to the model, the model may reflect the data and update the contribution of each reaction factor. In addition, when setting a specific experimental goal (e.g., specific yield or more, optimization in a specific reaction time, etc.), the model may find an optimal combination that satisfies the goal.

The researcher may reduce unnecessary experiments and may design a more reliable experiment by using the explainable yield prediction model. The optimal reaction combination may be recommended according to the experimental goal set by the researcher, and the optimization performance may be gradually improved by continuously training the model.

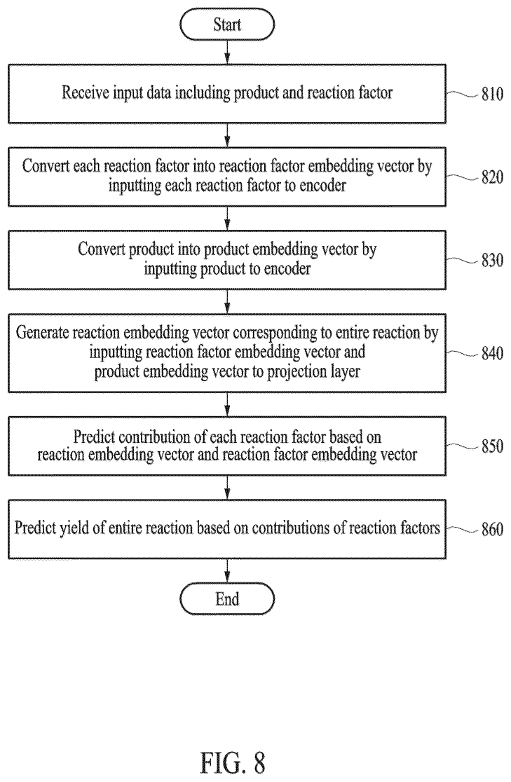

FIG. 8 is a flowchart illustrating an yield prediction model, according to an embodiment. The description provided with reference to FIGS. 1A, 1B, 2A, 2B, and 3 to 7 may apply to FIG. 8 in substantially the same manner.

For ease of description, operations 810 to 860 are described as being performed using the inference device 250 shown in FIG. 2B. However, operations 810 to 860 may be used by any other appropriate electronic device or in any appropriate system.

Furthermore, operations may be performed in the sequence and manner as illustrated in FIG. 8. However, some operations may be performed in a different order or may be omitted without departing from the spirit and scope of the described embodiments. The operations shown in FIG. 8 may be performed in parallel or simultaneously.

In operation 810, the inference device 250 in an embodiment may receive input data including a product and a reaction factor. The interference device 250 may include an input interface such as a physical keyboard, virtual keyboard, touchscreen, mouse, or microphone to receive the input data. The interference device 250 may also include a communication interface to connect to an external device, allowing it to receive the input data wirelessly or through a wired connection.

In operation 820, the inference device 250 in an embodiment may input the reaction factor to an encoder and may convert the reaction factor into a reaction factor embedding vector. The reaction factor may include a precursor and a reaction condition, and the inference device 250 may convert the precursor into a precursor embedding vector by inputting the precursor to a GNN encoder in a graph form. The inference device 250 may convert the reaction condition into a reaction condition embedding vector by inputting the reaction condition to an FNN encoder. The reaction condition embedding vector and the precursor embedding vector may be set to the same size.

In operation 830, the inference device 250 in an embodiment may convert the product into a product embedding vector by inputting the product to the encoder. The inference device 250 may convert the product into the product embedding vector by inputting the product to the GNN encoder in a graph form.

In operation 840, the inference device 250 in an embodiment may generate a reaction embedding vector corresponding to the entire reaction by inputting the reaction factor embedding vector and the product embedding vector to a projection layer. The inference device 250 may generate a reaction embedding vector by concatenating the reaction factor embedding vector with the product embedding vector and inputting the concatenated embedding vector to the projection layer.

In operation 850, the inference device 250 in an embodiment may predict the contribution of each reaction factor based on the reaction embedding vector and the reaction factor embedding vector. The inference device 250 may predict the contribution of each reaction factor by inputting the reaction factor embedding vector to a corresponding prediction head together with the reaction embedding vector.

In operation 860, the inference device 250 in an embodiment may predict the yield of the entire reaction based on the contribution of each reaction factor. The inference device 250 may predict the yield of the entire reaction by summing the contributions of reaction factors. The entire reaction may include at least one of a chemical reaction, an electrochemical reaction, a biochemical reaction, a material conversion process, a physical processing process, a data conversion process, or a manufacturing process.

The inference device 250 may predict the contribution of each reaction factor and the yield for a plurality of reaction factor combinations by differently setting the reaction factors and may determine the reaction factor according to a predefined criterion based on the predicted yield and contribution with respect to the plurality of reaction factor combinations.

The inference device 250 may include an output interface, such as a display screen or speaker, to present prediction results, including the contribution of each reactor factor and the overall yield of the reaction. Additionally, the inference device 250 may allow the user to set or adjust the reaction factors via the input interface, and subsequently provide updated prediction results reflecting the adjusted factors through the output interface.

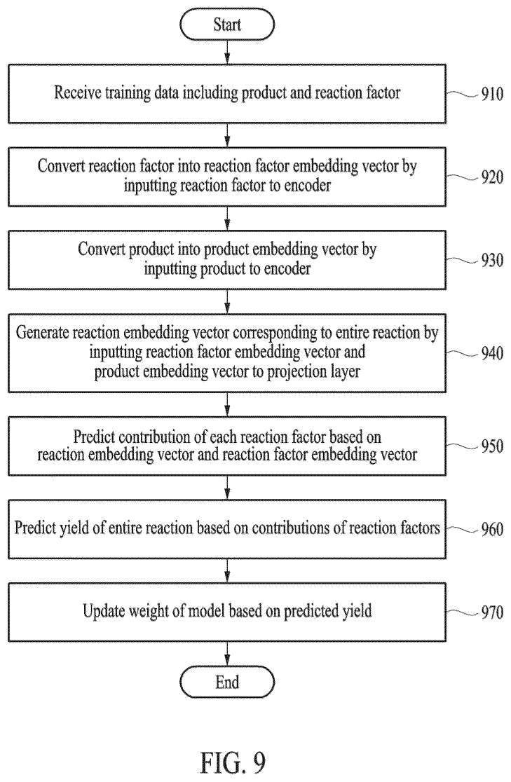

FIG. 9 is a flowchart illustrating a training method, according to an embodiment. The description provided with reference to FIGS. 1A, 1B, 2A, 2B, and 3 to 8 may apply to FIG. 9 in substantially the same manner.

For ease of description, operations 910 to 960 are described as being performed using the training device 200 shown in FIG. 2B. However, operations 910 to 960 may be used by any other appropriate electronic device or in any appropriate system.

Furthermore, operations may be performed in the sequence and manner as illustrated in FIG. 9. However, some operations may be performed in a different order or may be omitted without departing from the spirit and scope of the described embodiments. The operations shown in FIG. 9 may be performed in parallel or simultaneously.

In operation 910, the training device 200 in an embodiment may receive training data including a product and a reaction factor.

In operation 920, the training device 200 in an embodiment may convert the reaction factor into a reaction factor embedding vector by inputting the reaction factor to an encoder.

In operation 930, the training device 200 in an embodiment may convert the product into a product embedding vector by inputting the product to the encoder.

In operation 940, the training device 200 in an embodiment may generate a reaction embedding vector corresponding to the entire reaction by inputting the reaction factor embedding vector and the product embedding vector to a projection layer.

In operation 950, the training device 200 in an embodiment may predict the contribution of each reaction factor based on the reaction embedding vector and the reaction factor embedding vector.

In operation 960, the training device 200 in an embodiment may predict the yield of the entire reaction based on the contribution of each reaction factor.

In operation 970, the training device 200 in an embodiment may update a weight of the model based on the predicted yield. The training device 200 may update the weight of the model based on a first loss function that minimizes a difference between the predicted yield and ground truth yield, a second loss function that normalizes the sum of contributions of reaction factors to satisfy a predefined criterion, and a third objective function that corrects the contribution of a reaction factor that does not affect the yield.

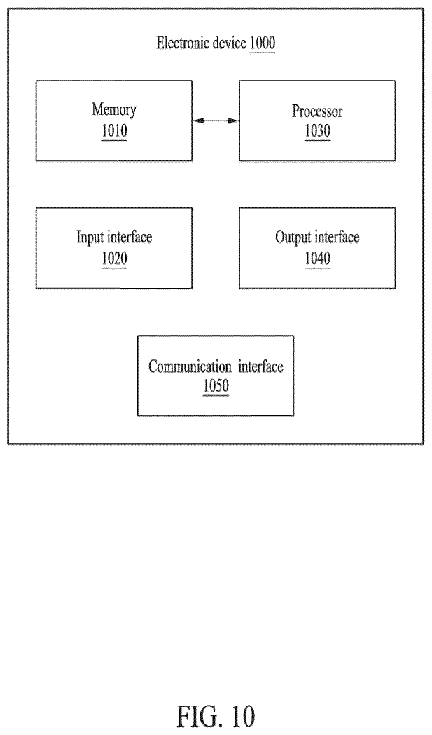

FIG. 10 illustrates an electronic device according to an embodiment. The description provided with reference to FIGS. 1A, 1B, 2A, 2B, and 3 to 9 may apply to FIG. 10 in substantially the same manner.

Referring to FIG. 10, an electronic device 1000 may include a memory 1010 and a processor 1030. The electronic device 1000 in an embodiment may include the inference device 250 described with reference to FIG. 2B.

The memory 1010 may store instructions (or programs) executable by the processor 1030. For example, the instructions include instructions for performing the operation of the processor 1030 and/or an operation of each component of the processor 1030.

The memory 1010 may be implemented as a volatile memory device or a non-volatile memory device.

The volatile memory device may be implemented as dynamic random-access memory (DRAM), static random-access memory (SRAM), thyristor RAM (T-RAM), zero capacitor RAM (Z-RAM), or twin transistor RAM (TTRAM).

The non-volatile memory device may be implemented as electrically erasable programmable read-only memory (EEPROM), flash memory, magnetic RAM (MRAM), spin-transfer torque (STT)-MRAM, conductive bridging RAM (CBRAM), ferroelectric RAM (FeRAM), phase change RAM (PRAM), resistive RAM (RRAM), nanotube RRAM, polymer RAM (PoRAM), nano floating gate Memory (NFGM), holographic memory, a molecular electronic memory device, or insulator resistance change memory.

The processor 1030 may process data stored in the memory 1010. The processor 1030 may process data stored in the memory 1010. The processor 1030 may execute computer-readable code (for example, software) stored in the memory 1010 and instructions triggered by the processor 1030.

The processor 1030 may be a data processing device implemented by hardware including a circuit having a physical structure to perform desired operations. For example, the desired operations may include code or instructions in a program.

For example, the data processing device implemented by hardware may include a microprocessor, a central processing unit (CPU), a processor core, a multi-core processor, a multiprocessor, an application-specific integrated circuit (ASIC), and a field-programmable gate array (FPGA).

The processor 1030 may receive input data including a product and a reaction factor, may convert the reaction factor into a reaction factor embedding vector by inputting the reaction factor to an encoder, may convert the product into a product embedding vector by inputting the product to the encoder, may generate a reaction embedding vector corresponding to the entire reaction by inputting the reaction factor embedding vector and the product embedding vector to a projection layer, may predict the contribution of each reaction factor based on the reaction embedding vector and the reaction factor embedding vector, and may predict the yield of the entire reaction based on the contribution of each reaction factor. The processor 1030 may perform the operations described with reference to FIGS. 1A, 1B, 2A, 2B, and 3 to 9 in substantially the same manner. Accordingly a detailed description is omitted.

The electronic device 1000 may further include an input interface 1020, an output interface 1040, and a communication interface 1050. The input interface 1020 may be implemented as a physical keyboard, virtual keyboard, touchscreen, mouse, or microphone to receive input data such as final product information, reaction conditions, and precursors. The communication interface 1050 may be implemented as any one or any combination of a digital modem, a radio frequency (RF) modem, an antenna circuit, a Wi-Fi chip, and related software and/or firmware to connect the electronic device 1000 to an external device, allowing it to receive input data wirelessly or through a wired connection. The output interface 1040 may be implemented as a display screen or speaker to present prediction results, including the contribution of each reactor factor and the overall yield of the reaction. Additionally, the electronic device 1000 may allow the user to set or adjust the reaction factors via the input interface 1020 (or an external device connected via the communication interface 1050) and subsequently provide updated prediction results reflecting the adjusted factors through the output interface 1040. The output interface 1040 may display the graphs 500, 710, 720, and 730 shown in FIGS. 5 and 7.

The embodiments described herein may be implemented using a hardware component, a software component, and/or a combination thereof. A processing device may be implemented using one or more general-purpose or special-purpose computers, such as, for example, a processor, a controller and an arithmetic logic unit (ALU), a digital signal processor (DSP), a microcomputer, an FPGA, a programmable logic unit (PLU), a microprocessor, or any other device capable of responding to and executing instructions in a defined manner. The processing device may run an operating system (OS) and a software application that runs on the OS. The processing device also may access, store, manipulate, process, and create data in response to execution of the software. For purpose of simplicity, the description of a processing device is used as singular; however, one skilled in the art will appreciate that a processing device may include multiple processing elements and multiple types of processing elements. For example, a processing device may include multiple processors or a processor and a controller. In addition, different processing configurations are possible, such as parallel processors.

The software may include a computer program, a piece of code, an instruction, or combinations thereof, to independently or uniformly instruct or configure the processing device to operate as desired. Software and data may be embodied permanently or temporarily in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

Although the embodiments have been described with reference to the limited drawings, one of ordinary skill in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents.

Accordingly, other implementations are within the scope of the following claims.

What is claimed is:

1. A method of predicting yield, the method comprising:
receiving input data comprising a product and reaction factors;
obtaining reaction factor embedding vectors via an encoder based on the reactor factors;
obtaining a product embedding vector via the encoder based on the product;
obtaining a reaction embedding vector corresponding to an entire reaction via a projection layer based on the reaction factor embedding vectors and the product embedding vector;
predicting a contribution of each of the reaction factors based on the reaction embedding vector and the reaction factor embedding vectors; and
predicting yield of the entire reaction based on the contribution of each of the reaction factors.

2. The method of claim 1, wherein the predicting of the contribution of each of the reaction factors comprises predicting the contribution of each of the reaction factors by inputting the reaction embedding vector and the reaction factor embedding vectors to a prediction head.

3. The method of claim 1, wherein the predicting of the yield of the entire reaction comprises predicting the yield of the entire reaction by summing the contributions of the reaction factors.

4. The method of claim 1, wherein the encoder comprises a graph neural network (GNN) encoder,
the reaction factor comprises a precursor and a reaction condition, and
the obtaining of the reaction factor embedding vectors comprises converting the precursor into a precursor embedding vector by inputting the precursor to the GNN encoder in a graph form.

5. The method of claim 4, wherein the encoder comprises a fully connected neural network (FNN) encoder,
the obtaining of the reaction factor embedding vectors comprises converting the reaction condition into a reaction condition embedding vector by inputting the reaction condition to the FNN encoder, and
the reaction condition embedding vector and the precursor embedding vector are set to a same size.

6. The method of claim 4, wherein the obtaining of the product embedding vector comprises converting the product into the product embedding vector by inputting the product to the GNN encoder.

7. The method of claim 1, wherein the obtaining of the reaction embedding vector comprises obtaining a concatenated reaction factor embedding vectors by concatenating the reaction factor embedding vectors with the product embedding vector and inputting the concatenated the reaction factor embedding vectors with the product embedding vector to the projection layer.

8. The method of claim 1, wherein the entire reaction comprises at least one of a chemical reaction, an electrochemical reaction, a biochemical reaction, a material conversion process, a physical processing process, a data conversion process, or a manufacturing process.

9. The method of claim 1, further comprising:
predicting the contribution of each of the reaction factors and the yield with respect to a plurality of reaction factor combinations by differently setting the reaction factors; and
determining a reaction factor according to a predefined criterion based on a predicted contribution and yield with respect to the plurality of reaction factor combinations.

10. The method of claim 1, wherein the encoder and the projection layer is included in a neural network model, and
the method further comprises updating weights of the neural network model based on the predicted yield.

11. The method of claim 10, wherein the updating of the weights of the neural network model comprises updating the weights of the neural network model based on a first loss function that minimizes a difference between the predicted yield and ground truth yield, a second loss function that normalizes a sum of the contributions of the reaction factors to satisfy a predefined criterion, and a third objective function that corrects a contribution of a reaction factor that does not affect the yield.

12. An electronic device comprising:
at least one processor comprising processing circuitry; and
memory storing instructions,
wherein the instructions, when executed individually or collectively by the at least one processor, cause the electronic device to:
receive input data comprising a product and reaction factors,
obtain reaction factor embedding vectors via an encoder based on the reactor factors,
obtain a product embedding vector via the encoder based on the product,
obtain a reaction embedding vector corresponding to an entire reaction via a projection layer based on the reaction factor embedding vectors and the product embedding vector,
predict a contribution of each of the reaction factors based on the reaction embedding vector and the reaction factor embedding vectors, and
predict yield of the entire reaction based on the contribution of each of the reaction factors.

13. The electronic device of claim 12, wherein the instructions, when executed individually or collectively by the at least one processor, cause the electronic device to predict the contribution of each of the reaction factors by inputting the reaction embedding vector and the reaction factor embedding vectors to a prediction head.

14. The electronic device of claim 12, wherein each sentence comprises a plurality of feature vectors, and
the instructions, when executed individually or collectively by the at least one processor, cause the electronic device to predict the yield of the entire reaction by summing the contributions of the reaction factors.

15. The electronic device of claim 12, wherein the encoder comprises a fully connected neural network (FNN) encoder, and the reaction factor comprises a precursor and a reaction condition, and
the instructions, when executed individually or collectively by the at least one processor, cause the electronic device to convert the reaction condition into a reaction condition embedding vector by inputting the reaction condition to the FNN encoder.

16. The electronic device of claim 15, wherein the instructions, when executed individually or collectively by the at least one processor, cause the electronic device to convert the reaction condition into the reaction condition embedding vector by inputting the reaction condition to the FNN encoder.

17. The electronic device of claim 15, wherein the instructions, when executed individually or collectively by the at least one processor, cause the electronic device to convert the product into the product embedding vector by inputting the product to a graph neural network (GNN) encoder in a graph form.

18. The electronic device of claim 12, wherein the instructions, when executed individually or collectively by the at least one processor, cause the electronic device to generate the reaction embedding vector by concatenating the reaction factor embedding vectors with the product embedding vector and inputting the concatenated the reaction factor embedding vectors with the product embedding vector to the projection layer.

19. The electronic device of claim 12, wherein the entire reaction comprises at least one of a chemical reaction, an electrochemical reaction, a biochemical reaction, a material conversion process, a physical processing process, a data conversion process, or a manufacturing process.

20. The electronic device of claim 12, wherein the instructions, when executed individually or collectively by the at least one processor, cause the electronic device to:
predict the contribution of each of the reaction factors and the yield with respect to a plurality of reaction factor combinations by differently setting the reaction factors, and
determine a reaction factor according to a predefined criterion based on a predicted contribution and yield with respect to the plurality of reaction factor combinations.
